(19) United States
(12) Patent Application Publication
(10) Pub. No.: US 2021/0121676 A1
(43) Pub. Date: Apr. 29, 2021

(54) ADMINISTRATION SYSTEM FOR ADMINISTERING A MEDICAL TREATMENT LIQUID TO A PATIENT AND EQUIPPED WITH A PURGE TUBING

(71) Applicant: DORAN INTERNATIONAL, Toussieu (FR)

(72) Inventors: Philippe BUISSON, TOUSSIEU (FR); Brice FIORE, Gambsheim (FR)

(21) Appl. No.: 16/869,258

(22) Filed: May 7, 2020

(30) Foreign Application Priority Data
Oct. 25, 2019 (FR) 19/11988

(51) Int. Cl. A61M 39/10; A61M 39/24; A61M 39/20

(52) U.S. Cl. CPC A61M 39/105; A61M 2039/229; A61M 39/20; A61M 39/24

(57) ABSTRACT

The administration system including a stopcock including a fluid inlet path, a first fluid outlet path and a second fluid outlet path, the first fluid outlet path being equipped with a connecting member configured to be connected to an administration extension tube; a purge tubing including a first end fluidly connected to the second fluid outlet path and a second end configured to be fluidly connected to a collection container; and a non-return valve configured to enable a flow of fluid from the second fluid outlet path to the purge tubing and to prevent a flow of fluid from the purge tubing to the second fluid outlet path.

### CROSS REFERENCE TO RELATED APPLICATIONS

This application claims priority under 35 U.S.C. § 119 to the following French Patent Application No. 19/11988, filed on Oct. 25, 2019, the entire contents of which are incorporated herein by reference thereto.

### BACKGROUND

The present invention concerns an administration system for administering a medical treatment liquid to a patient.

An administration system comprises, in a known manner:
a stopcock comprising a fluid inlet path, a first fluid outlet path and a second fluid outlet path, the fluid inlet path being equipped with a connecting element configured to allow a connection of a supply container, such as a syringe, to the fluid inlet path, the first fluid outlet path being equipped with a connecting member configured to be connected to an administration extension tube which is fluidly connected to a catheter,
a purge tubing including a first end fluidly connected to the second fluid outlet path and a second end configured to be fluidly connected to a collection container, such as a flexible collection bag.

The method for using such an administration system comprises the following steps:
connecting an administration extension tube to the first fluid outlet path,
connecting the distal end of the administration extension tube to a catheter connected to a patient,
connecting a rinsing container, such as a rinsing syringe, containing a rinsing product, such as physiological saline solution or glucose, to the fluid inlet path of the stopcock,
positioning a manipulating member belonging to the stopcock in a position in which the stopcock enables a flow of fluid from the fluid inlet path to the second fluid outlet path,
purging the stopcock by injecting, into the latter, a sufficient amount of the rinsing product contained in the rinsing container,
collecting the rinsing product in a collection container, such as a flexible collection bag, connected to the second end of the purge tubing,
disconnecting the rinsing container,
connecting an administration container, such as an administration syringe, containing a medical treatment liquid to the fluid inlet path of the stopcock,
positioning the manipulating member belonging to the stopcock in a position in which the stopcock enables a flow of fluid from the fluid inlet path to the first fluid outlet path, and
injecting the medical treatment liquid to the catheter using the administration container.

When a particular treatment for the patient is stopped, the method of use further includes the following steps:
disconnecting the administration container,
reconnecting a rinsing container, such as a rinsing syringe, containing a rinsing product, such as physiological saline solution or glucose, to the fluid inlet path of the stopcock,
positioning the manipulating member belonging to the stopcock in a position in which the stopcock enables a flow of fluid from the fluid inlet path to the second fluid outlet path,
purging the stopcock by injecting, into the latter, a sufficient amount of the rinsing product contained in the rinsing container, collecting the rinsing product in a collection container, such as a flexible collection bag, connected to the second end of the purge tubing, and
disconnecting the rinsing container.

The different aforementioned purge steps ensure an effective cleaning of the stopcock, for a subsequent treatment of the patient with the same medical treatment liquid or another medical treatment liquid.

However, if the manipulating member of the stopcock is accidentally positioned by a member of the hospital staff in a position in which the stopcock enables a flow of fluid from the second fluid outlet path to the first fluid outlet path, a portion of the fluid contained in the purge tubing and/or in the collection container is likely to flow into the extension tube and therefore to be administered to the patient, which can be detrimental to the patient.

### BRIEF DESCRIPTION

The present invention aims at overcoming all or part of these drawbacks. The technical problem underlying the invention therefore consists in providing an administration system which is of a simple, reliable and economic structure.

To this end, the present invention concerns an administration system for administering a medical treatment liquid to a patient, comprising:
a stopcock comprising a fluid inlet path, a first fluid outlet path and a second fluid outlet path, the fluid inlet path being equipped with a connecting element configured to allow a connection of a supply container, such as a syringe, to the fluid inlet path, the first fluid outlet path being equipped with a connecting member configured to be connected to an administration extension tube intended to be fluidly connected to a catheter,
a purge tubing including a first end fluidly connected to the second fluid outlet path and a second end configured to be fluidly connected to a collection container, such as a flexible collection bag, and
a non-return valve configured to enable a flow of fluid from the second fluid outlet path to the purge tubing and to prevent a flow of fluid from the purge tubing to the second fluid outlet path.

The presence of such a non-return valve avoids any risk of flow of a portion of the fluid, contained in the purge tubing and/or in the collection container, to an extension tube connected to the first fluid outlet path and also to a supply container connected to the fluid inlet path.

Thus, the administration system according to the present invention ensures a reliable administration of a medical treatment liquid to a patient.

The administration system may further have one or more of the following features, taken alone or in combination.

According to one embodiment of the invention, the non-return valve fluidly connects the second fluid outlet path to the purge tubing.

According to one embodiment of the invention, the non-return valve is disposed between the second fluid outlet path and the purge tubing.

According to one embodiment of the invention, the supply container is for example a syringe, a vial or an administration bag. Advantageously, the supply container includes a male coupling tip.

According to one embodiment of the invention, the non-return valve includes a closure member movable between a release position in which the closure member enables a flow of fluid from the second fluid outlet path to the purge tubing, and a closure position in which the closure member prevents a flow of fluid from the purge tubing to the second fluid outlet path.

According to one embodiment of the invention, the non-return valve includes a flow passage connecting the second fluid outlet path to the purge tubing, the closure member being configured to release the flow passage when the closure member occupies the release position, and being configured to close the flow passage when the closure member occupies the closure position.

According to one embodiment of the invention, the non-return valve includes a first fastening portion fastened to the second fluid outlet path and a second fastening portion fastened to the first end of the purge tubing.

According to one embodiment of the invention, the second fastening portion is equipped with a female luer-lock connecting tip.

According to one embodiment of the invention, the first fastening portion is equipped with a male luer connecting tip.

According to one embodiment of the invention, the administration system further comprises a coupling device coupled to the second end of the purge tubing and configured to fluidly connect the second end of the purge tubing to the collection container, the coupling device including a closure element which is deformable or displaceable between a first position in which the closure element prevents a flow of fluid through the coupling device, and a second position in which the closure element enables a flow of fluid through the coupling device, the closure element being configured to be deformed or displaced to the second position during a connection of the collection container to the coupling device.

According to one embodiment of the invention, the coupling device comprises a first coupling tip to which the collection container is intended to be coupled, and a second coupling tip, for example of the female luer-lock type, to which the second end of the purge tubing is coupled.

According to an embodiment of the invention, the connecting element is a female luer-lock connecting element.

According to one embodiment of the invention, the connecting member is a male luer-lock connecting member.

According to one embodiment of the invention, the administration system includes a protective cap configured to cooperate with and at least partially cover the connecting element equipping the fluid inlet path.

According to one embodiment of the invention, the administration system includes an additional protective cap configured to cooperate with and at least partially cover the connecting member equipping the first fluid outlet path.

According to one embodiment of the invention, the stopcock includes a manipulating member which is movable in rotation and which is configured to occupy a first position in which the fluid inlet path is fluidly connected to the first fluid outlet path, and a second position in which the fluid inlet path is fluidly connected to the second fluid outlet path.

According to one embodiment of the invention, the stopcock is a three-way stopcock.

According to one embodiment of the invention, the first end of the purge tubing is equipped with a first male luer-lock connecting tip, and the second end of the purge tubing is equipped with a second male luer-lock connecting tip.

### BRIEF DESCRIPTION OF THE DRAWINGS

In any case, the invention will be clearly understood using the following description, with reference to the appended schematic drawings representing, by way of non-limiting example, an embodiment of this administration system.

FIG. 1 is a schematic view of an administration system according to the present invention.

FIG. 2 is a schematic view of the administration system of FIG. 1 in a first configuration of use.

FIG. 3 is a schematic view of the administration system of FIG. 1 in a second configuration of use.

FIG. 4 is a schematic view of the administration system of FIG. 1 in a third configuration of use.

### DETAILED DESCRIPTION

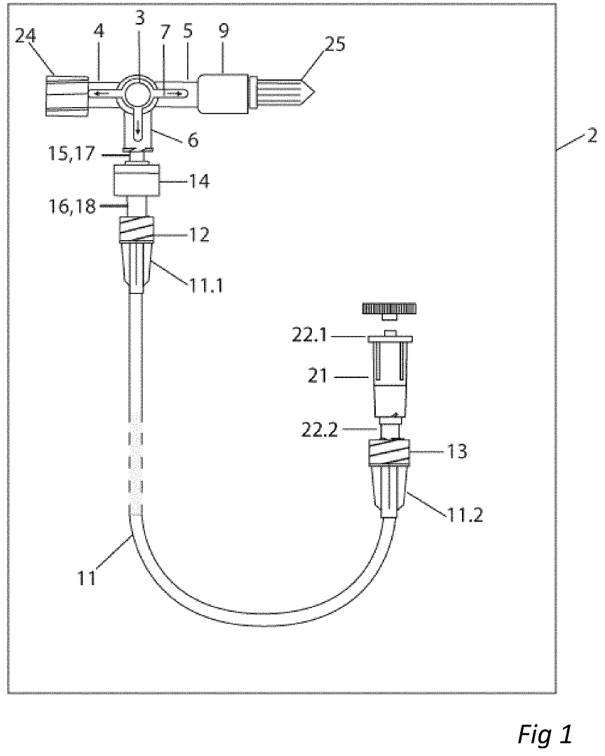

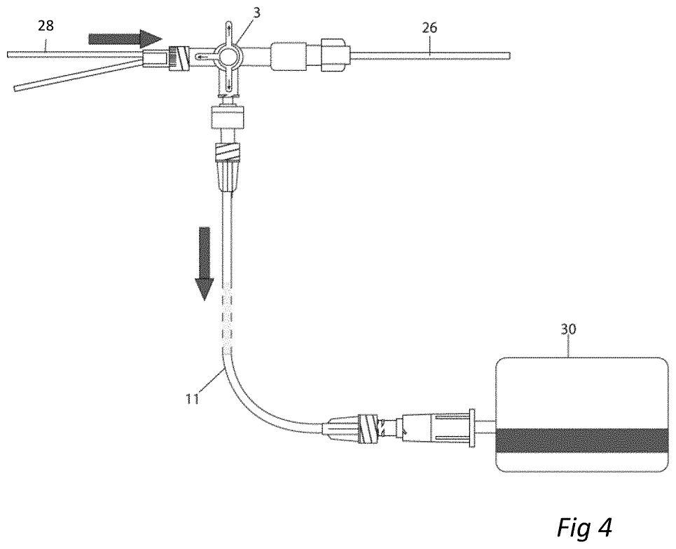

FIGS. 1 to 4 represent an administration system 2 for administering a medical treatment liquid to a patient.

The administration system 2 includes a three-way stopcock 3 comprising a fluid inlet path 4, a first fluid outlet path 5 and a second fluid outlet path 6.

The stopcock 3 includes a manipulating member 7 which is movable in rotation and which is configured to occupy a first position in which the fluid inlet path 4 is fluidly connected to the first fluid outlet path 5, and a second position in which the fluid inlet path 4 is fluidly connected to the second fluid outlet path 6.

The fluid inlet path 4 is equipped with a connecting element 8 configured to allow a connection of a supply container, such as a rinsing container or an administration container, a vial or even an administration bag, to the fluid inlet path 4. According to the embodiment represented in the figures, the connecting element 8 is a female luer-lock connecting element.

The first fluid outlet path 5 is equipped with a connecting member 9 configured to be connected to an administration extension tube intended to be fluidly connected to a catheter. According to the embodiment represented in the figures, the connecting member 9 is a male luer-lock connecting member.

The administration system 2 further includes a purge tubing 11 including a first end 11.1 fluidly connected to the second fluid outlet path 6 and a second end 11.2 configured to be fluidly connected to a collection container, such as a flexible collection bag. According to the embodiment represented in the figures, the first end 11.1 of the purge tubing 11 is equipped with a first male luer-lock connecting tip 12, and the second end 11.2 of the purge tubing 11 is equipped with a second male luer-lock connecting tip 13.

The administration system 2 also includes a non-return valve 14 which is disposed between the second fluid outlet path 6 and the purge tubing 11, and which is configured to enable a flow of fluid from the second fluid outlet path 6 to the purge tubing 11 and to prevent a flow of fluid from the purge tubing 11 to the second fluid outlet path 6.

The non-return valve 14 includes a first fastening portion 15 fastened to the second fluid outlet path 6 and a second fastening portion 16 fastened to the first end 11.1 of the purge tubing 11. According to the embodiment represented in the figures, the first fastening portion 15 is equipped with a male luer connecting tip 17, and the second fastening portion 16 is equipped with a female luer-lock connecting tip 18.

Advantageously, the non-return valve 14 includes a flow passage fluidly connecting the second fluid outlet path 6 to the purge tubing 11, and a closure member movable between a release position, in which the closure member frees the flow passage and enables a flow of fluid from the second fluid outlet path 6 to the purge tubing 11, and a closure position, in which the closure member closes the flow passage and prevents a flow of fluid from the purge tubing 11 to the second fluid outlet path 6.

The administration system 2 further comprises a coupling device 21 configured to fluidly connect the second end 11.2 of the purge tubing 11 to the collection container.

The coupling device 21 includes a closure element which is deformable or displaceable between a first position in which the closure element prevents a flow of fluid through the coupling device 21, and a second position in which the closure element enables a flow of fluid through the coupling device 21. The closure element is more particularly configured to be deformed or displaced to the second position when the collection container is connected to the coupling device 21. The closure element can for example be split and be elastically deformable. The edges delimiting the slot of the closure element are for example configured to elastically deviate from each other when the collection container is connected to the coupling device 21, and for example when a coupling portion equipping the collection container is inserted into the coupling device 21, so as to allow a flow of fluid through the slot and therefore through the coupling device 21, and to elastically get closer to each other when the collection container is disconnected from the coupling device 21 so as to prevent a flow of fluid through the slot and therefore through the coupling device 21.

According to the embodiment represented in the figures, the coupling device 21 comprises a first coupling tip 22.1 to which the collection container is intended to be coupled, and a second coupling tip 22.2, for example of the female luer-lock type, to which the second end 11.2 of the purge tubing 11 is coupled.

The administration system 2 also includes a protective cap 24 configured to cooperate with and at least partially cover the connecting element 8 equipping the fluid inlet path 4, and an additional protective cap 25 configured to cooperate with and at least partially cover the connecting member 9 equipping the first fluid outlet path 5.

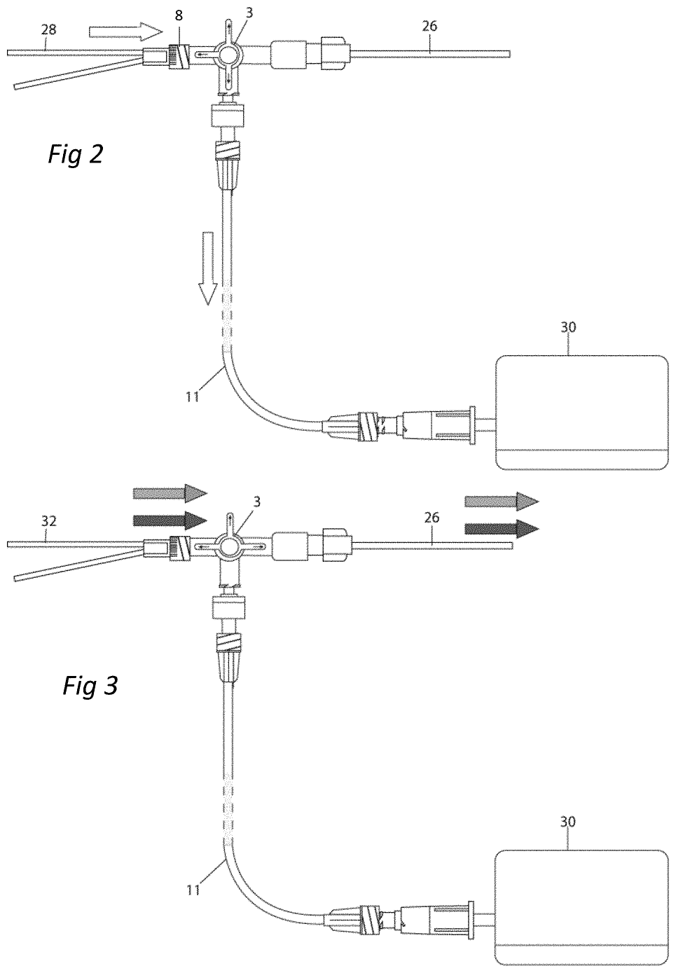

A method for using such an administration system 2 will now be described with reference to FIGS. 2 to 4. Such a method of use comprises the following steps:
connecting an administration extension tube 26 to the first fluid outlet path 5,
connecting the distal end of the administration extension tube 26 to a catheter connected to a patient,
connecting a rinsing container 28, such as a rinsing syringe or a rinsing bag, containing a rinsing product, such as physiological saline solution or glucose, to the fluid inlet path 4 of the stopcock 3 directly or via a connecting tubing,
positioning the manipulating member 7 belonging to the stopcock 3 in the position in which the stopcock 3 enables a flow of fluid from the fluid inlet path 4 to the second fluid outlet path 6,
purging the stopcock 3 by injecting, into the latter, a sufficient amount of the rinsing product contained in the rinsing container 28,
collecting the rinsing product in a collection container 30, such as a flexible collection bag, connected to the second end 11.2 of the purge tubing 11,
disconnecting the rinsing container 28,
connecting an administration container 32, such as an administration syringe or an administration bag, containing a medical treatment liquid to the fluid inlet path 4 of the stopcock 3 directly or via a connecting tubing,
positioning the manipulating member 7 belonging to the stopcock 3 in the position in which the stopcock 3 enables a flow of fluid from the fluid inlet path 4 to the first fluid outlet path 5, and
injecting the medical treatment liquid to the catheter using the administration container 32.

When a particular treatment for the patient is stopped, the method of use further includes the following steps:
disconnecting the administration container 32,
reconnecting a rinsing container 28, such as a rinsing syringe or a rinsing bag, containing a rinsing product, such as physiological saline solution or glucose, to the fluid inlet path 4 of the stopcock 3,
positioning the manipulating member 7 belonging to the stopcock 3 in the position in which the stopcock 3 enables a flow of fluid from the fluid inlet path 4 to the second fluid outlet path 6,
purging the stopcock 3 by injecting, into the latter, a sufficient amount of the rinsing product contained in the rinsing container 28,
collecting the rinsing product in a collection container 30 connected to the second end 11.2 of the purge tubing 11, and
disconnecting the rinsing container 28.

Given the presence of the non-return valve 14, any risk of flow of a portion of the fluid, contained in the purge tubing 11 and/or in the collection container 30, to an administration extension tube 26 connected to the first fluid outlet path 5 is avoided. Thus, the administration system 2 according to the present invention ensures a reliable administration of a medical treatment liquid to a patient.

Of course, the invention is not limited to the only embodiment of this administration system, described hereinabove by way of example, it encompasses, on the contrary, all variant thereof.

What is claimed is:

1. An administration system for administering a medical treatment liquid to a patient, comprising:
a stopcock comprising a fluid inlet path, a first fluid outlet path and a second fluid outlet path, the fluid inlet path being equipped with a connecting element configured to allow a connection of a supply container to the fluid inlet path, the first fluid outlet path being equipped with a connecting member configured to be connected to an administration extension tube intended to be fluidly connected to a catheter,
a purge tubing including a first end fluidly connected to the second fluid outlet path and a second end configured to be fluidly connected to a collection container, and
a non-return valve configured to enable a flow of fluid from the second fluid outlet path to the purge tubing and to prevent a flow of fluid from the purge tubing to the second fluid outlet path.

2. The administration system according to claim 1, wherein the non-return valve includes a first fastening portion fastened to the second fluid outlet path and a second fastening portion fastened to the first end of the purge tubing.

3. The administration system according to claim 1, which further comprises a coupling device coupled to the second end of the purge tubing and configured to fluidly connect the second end of the purge tubing to the collection container, the coupling device including a closure element which is deformable or displaceable between a first position in which the closure element prevents a flow of fluid through the coupling device, and a second position in which the closure element enables a flow of fluid through the coupling device, the closure element being configured to be deformed or displaced to the second position when connecting the collection container to the coupling device.

4. The administration system according to claim 3, wherein the coupling device comprises a first coupling tip to which the collection container is intended to be coupled, and a second coupling tip to which the second end of the purge tubing is coupled.

5. The administration system according to claim 1, wherein the connecting element is a female luer-lock connecting element.

6. The administration system according to claim 1, wherein the connecting member is a male luer-lock connecting member.

7. The administration system according to claim 1, which includes a protective cap configured to cooperate with and at least partially cover the connecting element equipping the fluid inlet path.

8. The administration system according to claim 1, which includes an additional protective cap configured to cooperate with and at least partially cover the connecting member equipping the first fluid outlet path.

9. The administration system according to claim 1, wherein the stopcock includes a manipulating member which is movable in rotation and which is configured to occupy a first position in which the fluid inlet path is fluidly connected to the first fluid outlet path, and a second position in which the fluid inlet path is fluidly connected to the second fluid outlet path.

10. The administration system according to claim 1, wherein the stopcock is a three-way stopcock.
